(19) United States
(12) Patent Application Publication
(10) Pub. No.: US 2006/0035388 A1
(43) Pub. Date: Feb. 16, 2006

(54) High-sensitivity magnetic marker used for immune response measurement

(75) Inventors: Kohji Yoshinaga, Kitakyushu-shi, Fukuoka (JP); Keiji Enpuku, Fukuoka-shi, Fukuoka (JP)

Correspondence Address: Jay R. Moldovanyi;Fay Sharpe Fagan Minnich & McKee, 1100 Superior Avenue, 7th Floor, Cleveland, OH, 44114-2518, US

(73) Assignee: Japan Science and Technology Agency, Saitama (JP)

(21) Appl. No.: 10/534,262

(22) PCT Filed: May 8, 2003
(86) PCT No.: PCT/JP03/05733

§ 371 Date: May 6, 2005

(30) Foreign Application Priority Data
Nov. 8, 2002 (JP) 2002-325026

(51) Int. Cl. G01N 33/553

(52) U.S. Cl. 436/526

(57) ABSTRACT

Disclosed are a highly-sensitive magnetic marker for use in measuring an immunoreaction with a SQUID magnetic sensor and the preparation thereof. The magnetic marker is composed of a magnetic fine particle and a polymer encapsulating the particle, wherein the particle diameter of the magnetic fine particle (preferably ferrite Fe₃O₄) is 20 to 40 nm and the external the diameter of the magnetic marker is 40 to 100 nm, the polymer having carboxyl groups on the surface thereof. The magnetic maker for a SQUID magnetic sensor is prepared by causing the surface of a magnetic fine particle to adsorb a hydrophilic macromonomer (preferably polyvinylpyrrolidone) having a polymerizable vinyl group at the terminal thereof and having a molecular weight of 500 to 1000, and then adding a monomer of a hydrophilic vinyl compound having carboxyl group and a crosslinking agent for carrying out copolymerization reaction.

### TECHNICAL FIELD

The present invention belongs to the technical field of immunoreaction measurement, and particularly relates to a highly-sensitive magnetic marker for use in measuring an immunoreaction utilizing a SQUID magnetic sensor.

### BACKGROUND ART

Measurement of an immunoreaction, i.e. an antigen-antibody reaction, is widely applied in various areas such as in the detection of germs or microbes, disease diagnosis, gene analysis, measurement of environmental substances and so on. An immunoreaction determination is done by measuring the specific binding of a target substance (antigen) with a test reagent (antibody) so as to qualify and/or quantify the target substance.

Hitherto immunoreaction measurement has been primarily conducted by means of the spectroscopic method: A test reagent (antibody) is provided with a spectroscopic marker such as a fluorescence-labeled enzyme, and an immunoreaction (antigen-antibody) reaction is detected by measuring the light emitted from the marker. However, while there is increasing demand for highly-sensitive and rapid detection of a microchemical reactions, the existing systems do not meet the requirements in many cases. Thus, there is a desire for a new type of immunoreaction measurement system with high sensitivity.

Recently, the SQUID (superconducting quantum interference device) has received considerable attention as a highly-sensitive magnetic sensor, as it enables the measurement of a very week magnetic field by taking advantage of a quantum effect (the quantization of magnetic flux). The most significant application of the SQUID is the measurement of the brain magnetic field, in which a magnetic field generated by the brain is measured to analyze or diagnose brain function. Other applications have started to emerge in various areas such as medical science, material evaluation, material analysis, precision measurement, natural resources survey and so on, and it is also proposed to apply the SQUID to immunoreaction measurement (cf. for example, K. Enpuku “Antigen-antibody reaction measurement utilizing SQUID,” Ouyou-butsuri, Vol. 70, No. 1, p48-49 (2001)).

In an immunoreaction measurement system utilizing the SQUID, an antibody material is attached to the surface of a magnetic marker composed of a polymer encapsulating a fine magnetic material. An antigen-antibody reaction will take place between the antibody and an antigen (the target substance) to produce a weak magnetic field signal attributable to the magnetic marker, which is measured by a SQUID (see FIG. 1). The general practice is to fix the SQUID and move the sample to be measured for the detection of a magnetic signal.

While the immunoreaction measurement system using the SQUID is a highly-sensitive sensor ascertained to be about ten times more sensitive than the fluorescent antibody method (see the reference mentioned above), further improvement is expected to produce a detection system for immunoreactions with still higher sensitivity. One approach to such improvement of the immunoreaction detection system using the SQUID is to develop an optimized magnetic marker while also improving instrumentation such by lowering noise (noise reduction).

However, there is found no prior art developed through a systematic study of the conditions to be met in improving the sensitivity of a magnetic marker for use in the SQUID magnetic sensor. For example, although reference is made in WO96/27133 (PCT/EP 96/00823) to magnetic particles for immunoassay including a magnetic sensor using the SQUID, there are no concrete disclosures of technologies for improving the sensitivity of a magnetic marker for use in the SQUID magnetic sensor. It is mentioned that the size of the magnetic particles ranges widely from 1 to 1000 nm, but this can be considered to be an arbitrary definition not based on technical studies into magnetic particle size. Moreover, no concrete disclosures are found on the type of polymers for obtaining a highly-sensitive magnetic marker or the production thereof.

The magnetically labeled antibody discussed above, in which a magnetic particle is encapsulated within a polymer and an antibody is bound to the surface of the polymer, has primarily been used to the purification and separation of antibodies. When commercially available magnetic particles are used for this purpose, the diameter of the magnetic particle is about 10 to 15 nm, while the size of polymer particle (i.e. the external diameter of the assembly as a whole) is 50 to 1000 nm. However, such conventional magnetically labeled antibodies cannot be applied to detect an antigen-antibody reaction with high sensitivity, because the properties of the magnetic particles are insufficient for such applications.

The object of the present invention is to provide a novel technology relating to a highly-sensitive magnetic marker suitable for use in the measurement of an immunoreaction using the SQUID magnetic sensor.

### DISCLOSURE OF INVENTION

After extensive studies, the present inventors achieved the present invention by noting that the size of the magnetic fine particle composing the core of the magnetic marker, and also the size of the polymer particle encapsulating the magnetic fine particle (more strictly, the external diameter of the magnetic marker as a whole), are parameters that affect the sensitivity of a magnetic marker for the SQUID magnetic sensor, and they successfully designed a polymer synthesizing system that ensures the preparation of a magnetic marker in which these parameters are optimized.

Thus, according to the present invention there is provided a magnetic marker composed of a magnetic fine particle and a polymer encapsulating the particle, for use in measuring an immunoreaction with a SQUID magnetic sensor, wherein the particle diameter of said magnetic fine particle is 20 to 40 nm and the diameter (external diameter) of said magnetic marker is 40 to 100 nm, said polymer having carboxyl groups on the surface thereof. In a preferred embodiment of the magnetic marker of the present invention, the magnetic fine particle is composed mostly of Fe₃O₄.

The present invention also provides a method for preparing the above-mentioned magnetic marker for use in a SQUID magnetic sensor, which comprises the steps of (i) causing the surface of a magnetic fine particle to adsorb a hydrophilic macromonomer having a polymerizable vinyl group at the terminal thereof and having a molecular weight of 500 to 1000, and then (ii) adding a monomer of hydrophilic vinyl compound having carboxyl groups and a crosslinking agent for carrying out a copolymerization reaction. In a preferred embodiment of the method for preparing the magnetic marker for a SQUID magnetic sensor according to the present invention, the macromonomer for use in the synthesis of the polymer is polyvinylpyrrolidone, polyoxyethylene or polyacrylamide.

### BRIEF DESCRIPTION OF THE DRAWINGS

FIG. 1 schematically shows the principle of measuring an immunoreaction by a SQUID magnetic sensor using the magnetic marker of the present invention.

FIG. 2 illustrates a reaction scheme according to the present invention, in which a magnetic fine particle is encapsulated (coated) with a polymer, as well as the chemical formulae of the reactants used in the reaction.

FIG. 3 shows an example of the adsorption isotherms in the case where a magnetic fine particle is made to adsorb a macromonomer according to the present invention.

FIG. 4 shows an example of the particle diameter distribution of particles (magnetic markers) obtained by encapsulating (coating) magnetic fine particles with a polymer according to the present invention.

FIG. 5 shows an electromicroscopic (SEM) view of an unmodified ferrite fine particle prior to the polymer-encapsulation (polymer-coating) according to the present invention.

FIG. 6 shows an electromicroscopic (SEM) view of a composite particle (a magnetic marker) prepared by the polymer-encapsulation (polymer-coating) according to the present invention.

FIG. 7 graphically shows an example of the results obtained when an antibody was adsorbed onto a magnetic marker of the present invention.

FIG. 8 shows an example of the relationship between the weight of the magnetic fine particle contained in the magnetic marker of the present invention and the SQUID output.

FIG. 9 shows an example of the results of protein detection experiments using an antibody-bound magnetic marker of the present invention, illustrating the relationship between the quantity of the protein and the SQUID output.

### BEST MODE FOR CARRYING OUT THE INVENTION

The present invention emerged from step-by-step studies carried out to determine the dominant factors affecting the sensitivity of a magnetic marker for use in a SQUID magnetic sensor and culminated in the attainment of an extremely highly-sensitive magnetic marker. The embodiments of the present invention will be detailed below with reference to such factors.

(1) Magnetic Fine Particle and its Size:

The present inventors discovered that the size (the diameter) of a magnetic fine particle encapsulated by a polymer to be applied as a magnetic marker for use in a SQUID magnetic sensor should be larger than that of the commercially available magnetic fine particle mentioned previously; that is to say, the diameter is required to be 20 to 40 nm. This is because the magnetic signal from the magnetic fine particle is proportional to the volume of the fine particle, and hence a larger particle will produce a larger magnetic signal. The increase in the volume of the magnetic fine particle will also induce a change in the magnetic characteristics: A small particle will exhibit so-called superparamagnetism whereas a large particle will exhibit residual magnetism. This also contributes to the enhancement of the magnetic signal.

The minimal diameter for a particle to develop magnetism as mentioned above is supported by a theoretical calculation as follows: When the volume of a magnetic fine particle is represented by V and the magnetic anisotropy energy thereof is represented by K, the transition from superparamagnetism to residual magnetism takes place at the point defined by the equation KV/k_{B}T=20, where k_{B} is Boltzmann constant and T is 300K. In the case of using Fe₃O₄ as the magnetic fine particle, it is estimated that K=10 to 20 (kJ/m³). This corresponds to a diameter of the fine particle of d=20 to 25 nm. It can thus be seen that the size of the magnetic fine particle is desirably d>20 nm.

It is crucial for a magnetically labeled antibody, to which the present invention is directed, to possess a sufficient dispersibility, since the antibody is used so as to bind with an antigen (a target substance to be measured) in an aqueous medium. Poor dispersibility will inhibit the antigen-antibody reaction. If the size of the magnetic fine particle is too large, significant sedimentation of the particles will occur along with poor dispersibility. In order to avoid these issues, it is necessary for the specific gravity of the polymer for encapsulating the magnetic fine particle (more strictly, the specific gravity of the magnetic marker) as a whole to be kept at approximately 1 to 3. This also means that the size of the magnetic fine particle is required to be d<40 nm.

While any of various materials can be used as the magnetic fine particle, including magnetite, Fe₂O₃ and Fe₃O₄, ferrite Fe₃O₄ is most preferably used since it exhibits the maximal magnetism.

(2) External Diameter of Magnetic Marker:

In a magnetic marker for a SQUID magnetic sensor of the present invention, it is also essential for the diameter of the polymer particle (more strictly, the external diameter of the magnetic marker as a whole) to be 40 nm or larger and 100 nm or smaller. This is because, if the polymer size is too large in the detection of an immunoreaction (an antigen-antibody binding reaction), the binding between the magnetically labeled antibody and the antigen will not proceed efficiently. A magnetic marker having too-large particle size (external diameter) is also undesirable because of poor dispensability that readily causes sedimentation, as mentioned above with reference to the magnetic fine particle.

(3) Polymer to be Used:

The magnetic marker for a SQUID magnetic sensor having the above-mentioned characteristics can be optimally prepared by taking advantage of the polymer system designed by the present inventors. Specifically, according to the present invention, the encapsulation or coating of a magnetic fine particle with a polymer can be effectively conducted by causing the surface of the magnetic fine particle to adsorb a hydrophilic macromonomer having a polymerizable vinyl group at the terminal thereof and having a molecular weight of 500 to 1000, and then adding a monomer of hydrophilic vinyl compound having carboxyl groups and a crosslinking agent for carrying out a copolymerization reaction, thereby producing the magnetic marker for a SQUID sensor, wherein the particle diameter of the magnetic fine particle is 20 to 40 nm and the external diameter of the magnetic marker is 40 to 100 nm, the polymer having carboxyl groups on the surface thereof.

While a particularly preferred example of the macromonomer for use is polyvinylpyrrolidone, other polymers such as polyoxyethylene or polyacrylamide can be employed. The adsorption of such a macromonomer onto the magnetic fine particle is generally carried out by dispersing magnetic fine particles, typified by ferrite Fe₃O₄, in methanol, and adding the macromonomer into the dispersion, followed by stirring for several hours at room temperature.

The magnetic fine particles with the macromonomer adsorbed thereon are then dispersed in a low-polar solvent (e.g. tetrahydrofuran), so that the surface of the magnetic polymer particle is encapsulated or coated with the polymer through copolymerization (radical polymerization) of the crosslinking agent and the monomer. A trivinyl compound is generally used as the crosslinking agent. As the monomer, a vinyl compound is preferably used which possesses carboxyl groups and is a hydrophilic molecule as a whole. The use of a hydrophobic monomer with a long alkyl chain having no hydrophilic carboxyl groups will result in a magnetic marker exhibiting poor dispersibility.

Thus, the polymer system employed in the present invention is based on a quite novel technical idea of encapsulating or coating a magnetic fine particle. Regarding a magnetic material utilizing polyvinylpyrrolidone, a process is known in which a magnetic powdery material is admixed with a vinylpyrrolidone-vinyl acetate copolymer resin (Japanese Patent Application Publication No. 2000-28616). However, it is apparent that the method of the present invention is totally different from such process.

According to the present invention, it is possible to encapsulate a magnetic fine particle, such as of ferrite Fe₃O₄, homogeneously with the synthetic polymer to a uniform thickness, and what is more, the resultant magnetic fine particle-synthetic polymer composite is imparted with a desired amount of carboxyl groups on the surface thereof. Specifically, the polymer may have on the surface thereof 500 to 5000, desirably 2000 to 3000, carboxyl groups per particle of the magnetic marker.

According to the present invention, it is also possible to control the particle diameter of the magnetic marker so as to be in the range of 40 to 100 nm by adjusting the conditions in the respective steps of the method of the present invention, i.e., the steps of causing the surface of a magnetic fine particle to adsorb a macromonomer and then adding a monomer having carboxyl groups to enable reaction with a crosslinking agent for radical copolymerization. In addition, the method of the present invention makes it possible to encapsulate magnetic fine particles with the monomer having carboxyl groups individually without inducing aggregation among the p articles.

(4) Characteristic Properties of the Magnetic Marker:

The magnetic marker for a SQUID magnetic sensor of the present invention as prepared in the above-mentioned manner has excellent dispersibility, and the dispersion in an aqueous medium is stably retained generally for a period of longer than one month.

The magnetic marker of the present invention for a SQUID sensor possesses a number of carboxyl groups on the surface thereof, and hence is capable of binding antibodies thereon via the carboxyl groups. The antibody binding ability of the magnetic marker of the present invention is very high: For example, it was found to be capable of binding a rabbit IgG with a yield of 80% or more.

The magnetic marker of the present invention, when bound with an antibody, is subject to the process of measuring the immunoreaction (antigen-antibody reaction) as explained previously. The sensitivity in the measurement is extremely high: For example, it is possible to measure an antigen (a protein) down to even as little as 1 pg (picogram) or less.

### EXAMPLE

The present invention will be explained more specifically with reference to the following working examples, which are not for restricting the present invention.

### Example 1

### Preparation of Ferrite Fine Particle Encapsulated with Polymer

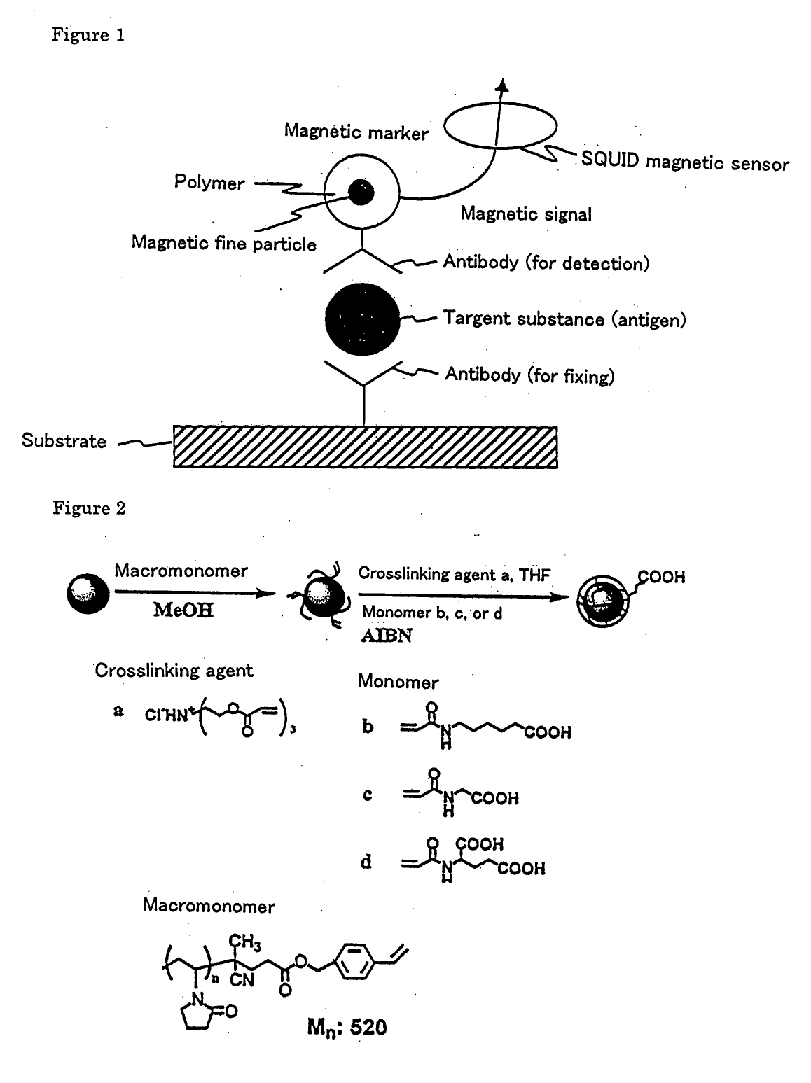

In accordance with the reaction schemes shown in FIG. 1, there were prepared polymer-encapsulated ferrite fine particles having carboxyl groups on the surface thereof (magnetic marker for a SQUID magnetic sensor).

<Adsorption of Polyvinylpyrrolidone onto Ferrite Fine Particle>

Into methanol 10 ml there was dissolved polyvinylpyrrolidone (molecular weight: 520), as the macromonomer, in an amount in the range of 0.004 to 0.04 g, followed by addition of fine particles of ferrite Fe₃O₄ 0.05 g (Toda-Kogyo Ltd., particle diameter: 25 nm) and then the resultant was subjected to ultrasonic irradiation. Following gentle stirring for four hours, the particles having polyvinylpyrrolidone adsorbed thereon were isolated with a centrifuge and then subjected to drying in vacuo.

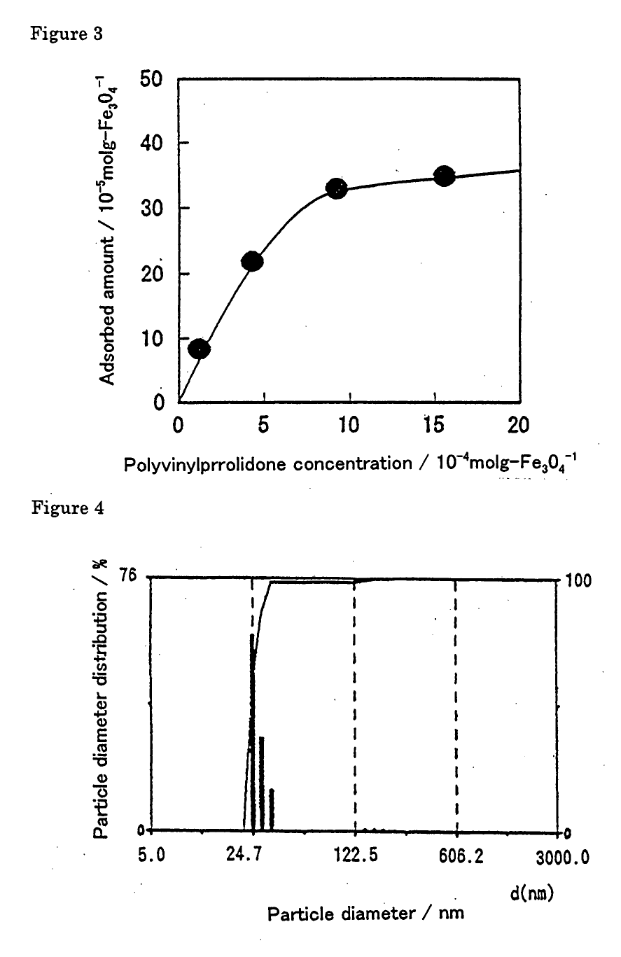

The amount adsorbed was calculated from the loss in weight during the process over a temperature rise from 100 to 800° C. FIG. 3 shows the adsorption isotherm. It was found that the amount of polyvinylpyrrolidone adsorbed leveled off when it reaches 1.0×10⁻³ mol per 1 gram of the ferrite particle.

<Polymer Encapsulation of Ferrite Fine Particle via Radical Copolymerization>

The amount adsorbed was calculated from the loss in weight during the process over a temperature rise from 100 to 800° C. FIG. 3 shows the adsorption isotherm. It was found that the amount of polyvinylpyrrolidone adsorbed leveled off when it reaches 1.0×10⁻³ mol per 1 gram of the ferrite particle.

<Polymer Encapsulation of Ferrite Fine Particle via Radical Copolymerization>

The ferrite fine particles having the hydrophilic macromonomer adsorbed thereon were dispersed in tetrahydrofuran, for polymer encapsulation or coating through the copolymerization between the crosslinking agent (trivinyl compound) and the monomer in the presence of AIBN (2,2′-azobis(isobutyronitrile)) (polymerization initiator), as detailed below.

<Encapsulation 1: Polymer-Encapsulation Around Ferrite Fine Particle Through the Copolymerization Between Tri(1-acryloyloxyethyl)amine Hydrochloride (a) and N-acryloyl-1-aminopentane (b)>

Into tetrahydrofuran 5 ml, there were dissolved N-acroylaminopentanate 0.12 g and 0 to 100 times in amount of tri(1-acryloyloxyethyl)amine hydrochloride, (tri (acroyloxy) ethylene) amine hydrochloride, as the crosslinking agent, in which the quantity of N-acroylaminopentanate was 100 times that of vinyl groups of the polyvinylpyrrolidone adsorbed onto the ferrite fine particle. To the resultant solution there were added ferrite fine particles 0.018 g and 2,2′-azobis(isobutyronitrile) 0.01 g, in which the ferrite particle had polyvinylpyrrolidone adsorbed thereon at the rate of polyvinylpyrrolidone 0.2 g per one gram of the ferrite particles. The solution was stirred at 65° C. for ten hours. The composite particles were separated from the solution with a centrifuge. The procedures were repeated five times, followed by separation of the unreacted monomer and crosslinking agent.

Table 1 shows the amounts of the polymer on the surfaces of the ferrite fine particles thus prepared. As shown in Table 1 the amount of the polymer bound increased with increasing amount of the crosslinking agent, whereas the fact that no aggregation occurred among the particles can be seen from the fact that the particle diameter was of the order of 29 to 30 nm as measured by the dynamic light scattering method (DLS). It should be noted that particle size measured by DLS is generally smaller than the actual size observed by a microscope, as will be explained later. The composite particles prepared in the above manner exhibited a relatively short period of retaining the dispersion, i.e. two days at the maximum. This is probably because the long methylene chain, which is hydrophobic, induces some degree of aggregation among the particles due to the low polar interaction in the aqueous medium.

TABLE 1

| | Cross linking | | Amount of | Particle | |
|---|---|---|---|---|---|
| | agent (a) | Monomer (b) | polymer | size | Dispersion |
| | 10⁻³ mol/g- | 10⁻³ mol/g- | bound | diameter | retaining |
| Entry | Fe₃O₄ | Fe₃O₄ | mg/g-Fe₃O₄ | nm | period |
| 1 | 0 | 37.7 | 527 | 32 | two days |
| 2 | 0.4 | 37.7 | 656 | 30 | Six hours |
| 3 | 3.7 | 37.7 | 706 | 30 | Six hours |
| 4 | 18.9 | 37.7 | 733 | 30 | Six hours |
| 5 | 37.7 | 37.7 | 866 | 29 | Six hours |

<Encapsulation 2: Polymer-Encapsulation Around Ferrite Fine Particle Through Copolymerization Between Tri(1-acryloyloxyethyl)amine Hydrochloride (a) and N-acryloylglycine (c)>

This encapsulation was carried out in the same manner as in Encapsulation 1 above. The results are given in Table 2. The amount of the polymer bound increased with increasing amount of the crosslinking agent, with a maximum value of approx. 870 mg/g. Of the composite particles, the particles having the polymer bound in an amount of 650 to 700 mg/g exhibited a particularly stable dispersion. with the dispersion in the aqueous medium being retained for longer than four weeks. The amount of the carboxyl groups on the surface also increased with increasing amount of the crosslinking agent, with a maximum value of 60 μmol/g. The particle diameter was measured by the DLS method.

TABLE 2

| | Cross-kinking | | Amount of | | Amount of | |
|---|---|---|---|---|---|---|
| | agent (a) | Monomer (c) | polymer | Particle | carboxyl | Dispersion |
| | 10⁻³ mol/g- | 10⁻³ mol/g- | bound | diameter | groups | retention |
| Entry | Fe₃O₄ | Fe₃O₄ | mg/g-Fe₃O₄ | nm | μmol/g | period |
| 1 | 0 | 39.8 | 527 | 34 | 26.6(1050) | 4 weeks |
| 2 | 0.4 | 39.8 | 656 | 26 | 28.6(1200) | Longer than |
| | | | | | | 4 weeks |
| 3 | 4.0 | 39.8 | 706 | 29 | 41.4(1800) | Longer than |
| | | | | | | 4 weeks |
| 4 | 19.9 | 39.8 | 733 | 25 | 51.9(2350) | 2 weeks |
| 5 | 39.8 | 39.8 | 866 | 33 | 59.7(2900) | 2 weeks |

The numerical values in ( ) indicate the number of carboxyl groups calculated as being present on the polymer surface per particle.

<Encapsulation 3: Polymer-Encapsulation Around Ferrite Fine Particle Through Copolymerization Between Tri(1-acryloyloxyethyl)amine Hydrochloride (a) and N-acryloylglutamic Acid (d)>

The encapsulation was carried out in the same manner as in Encapsulation 1. The results are given in Table 3. The particle diameter as shown in the table was measured by the DLS method. In this encapsulation it was also found that there occurred no aggregation among the particles and that the amount of the polymer bound increased with increasing amount of the crosslinking agent, with a maximum value of 947 mg/g. FIG. 4 shows the particle-diameter distribution (measured by the DLS method) of the composite particle given as Entry 4 in Table 4. It can be seen that there were no particles of a large diameter due to the aggregation. The amount of the carboxyl groups on the surface also increased with increasing the amount of the crosslinking agent, with a maximum value of 97 mmol/g. This value corresponds to 0.7 carboxyl groups per square nanometer of the surface of the particle. All the composite particles prepared by this encapsulation produced a stable dispersion in the aqueous medium, which was retained for a period of longer than four weeks.

TABLE 3

| | Cross-linking | | Amount of | | Amount of | |
|---|---|---|---|---|---|---|
| | agent (a) | Monomer (d) | polymer | Particle | carboxyl | Dispersion |
| | 10⁻³mol/g- | 10⁻³mol/g- | bound | diameter | groups | retention |
| Entry | Fe₃O₄ | Fe₃O₄ | mg/g-Fe₃O₄ | nm | μmol/g | period |
| 1 | 0 | 31.7 | 433 | 34 | 37.8(1400) | Longer than |
| 2 | 0.3 | 31.7 | 476 | 30 | 47.5(1750) | 4 weeks |
| 3 | 3.2 | 31.7 | 648 | 26 | 73.6(3100) | |
| 4 | 15.8 | 31.7 | 871 | 27 | 92.7(4500) | |
| 5 | 31.7 | 31.7 | 947 | 27 | 97.2(4850) | |

The numerical values in ( ) indicate the number of carboxyl groups calculated as being present on the polymer surface per particle.

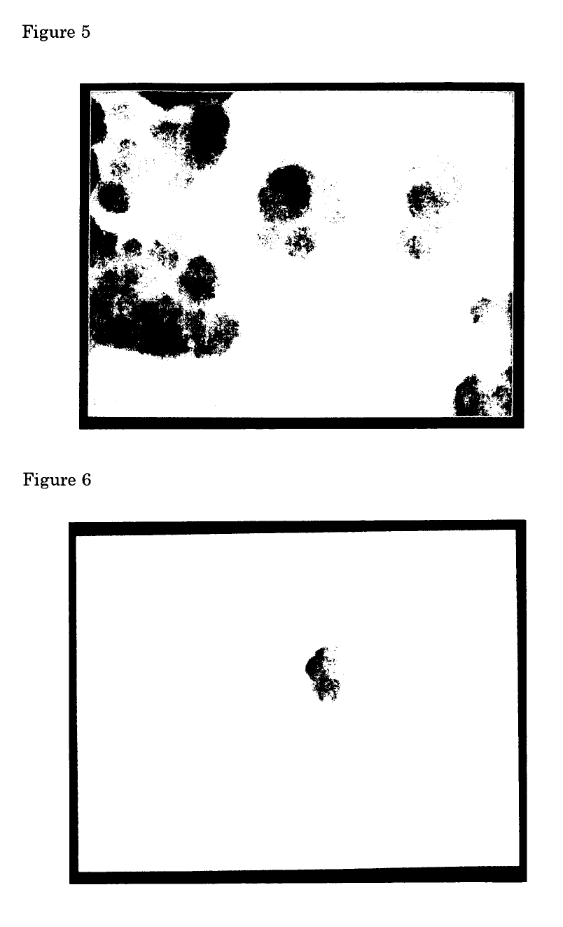

FIG. 5 and FIG. 6 are microscopic (SEM) views of unmodified ferrite fine particles and polymer-encapsulated (Encapsulation 3) ferrite fine particles, respectively. It is seen from the SEM view of the unmodified ferrite fine particles that aggregation was produced among the particles during the drying process in the preparation of the sample, whereas it was confirmed that the polymer-encapsulated particles developed excellent dispersion with the diameter (the external diameter) of the particle being about 80 nm.

The amount of carboxyl groups on the polymer surface as shown in Table 2 and Table 3 was determined as follows: To dehydrated, distilled chloroform 5 ml, there were added composite particles (polymer-encapsulated ferrite fine particles) 10 mg and N,N′-dicyclohexylcarbodiimide 15 mg, followed by stirring for two hours at room temperature. To the resultant dispersion was added p-nitrophenol 15 mg, followed by stirring for twelve hours at room temperature. Following the separation of unreacted p-nitrophenol from the composite particles using a centrifuge, the particles were subjected to drying in vacuo. Then, the particles having the p-nitrophenolate groups thereon were weighed and dispersed in a 4% ammonia aqueous solution, followed by gentle stirring for twelve hours. The solution in which p-nitrophenol was liberated was isolated from the composite particles by centrifuging, and then the solutions were combined, giving a total volume of 10.0 ml. The amount of p-nitrophenol contained in the aqueous solution thus obtained was determined through the absorbance at 400 nm (molar absorptivity ε=18000).

### Example 2

### Binding of Antibody

Polymer-encapsulated ferrite particles 0.017 g (magnetic marker), prepared in the manner of Encapsulation 3 of Example 1, were dispersed in a phosphate buffer solution (pH 7.8) 5 ml, followed by the addition of 1-ethyl-3-(3-dimethylaminopropyl) carbodiimido hydrochloride 0.01 g. Following stirring of the resultant solution for one hour at 4° C., there was added 0.016 mg of rabbit antibody (9.3 mg per gram of the fine particles) and then the solution was stirred for six hours at room temperature. The particles having the antibody bound thereon were separated from the phosphate buffer solution using a centrifuge. The amount of the antibody bound was 7.0 mg/g. The amount of the antibody bound was calculated from the amount of the antibody fed minus the amount of the nonbound antibody, wherein the amounts were determined through the absorbance at 280 nm.

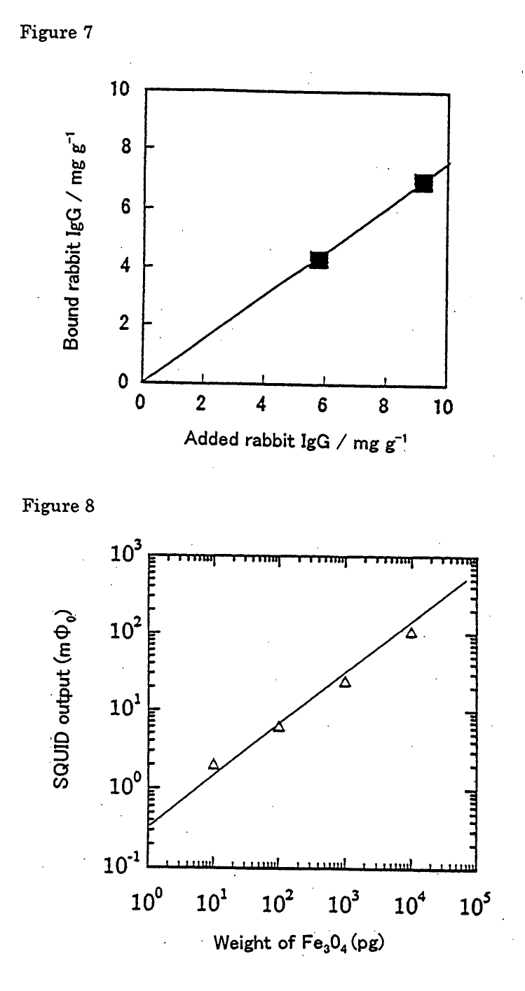

FIG. 7 shows the results of the binding of the IgG onto the polymer-encapsulated ferrite fine particles. In the case where about 10 mg of the antibody was added per gram of the particles, it is indicated that approx. 80% of the antibody was successfully bound onto the particles. It is thus evidenced that the composite particle (the magnetic marker) prepared according to the present invention exhibits a high ability of binding an antibody thereon.

### Example 3

### Relation Between Magnetic Material and SQUID Output

The magnetic signal from a magnetic marker of the present invention was measured by a SQUID magnetic sensor; the magnetic marker was composed of Fe₃O₄ fine particles with a diameter of 25 nm, as prepared by Encapsulation 3 of Example 1, and the polymer encapsulating the particle and having carboxyl groups on the surface thereof, and had an external diameter of 80 nm. FIG. 8 shows the results of the SQUID output measurements against varying weight of the magnetic marker. The ordinate is the weight of the ferrite fine particle (pg) in the magnetic marker while the abscissa is the SQUID output (mΦ₀). As can be seen from the figure, there is a good relationship between the weight of the marker and the SQUID output. As it is possible for a SQUID sensor to make a measurement to a level of 0.1 mΦ₀ or lower, the figure indicates that the magnetic marker of the present invention enables the measurement of the ferrite magnetic fine particle down to even an amount of less than 1 pg.

### Example 4

### Relation Between Antibody-Bound Magnetic Marker and SQUID Output

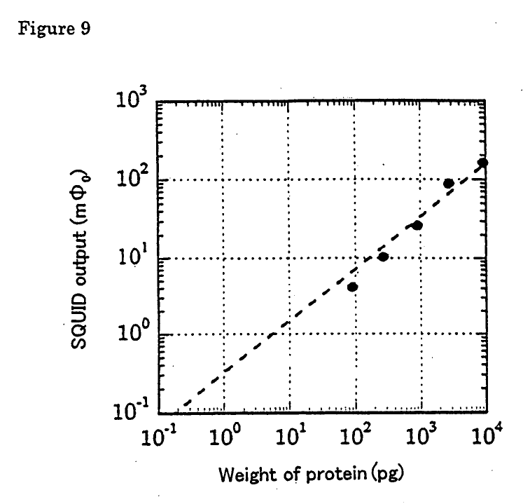

Detection of an antigen (protein) was carried out using the magnetic marker having the antibody bound thereon as prepared in Example 2, together with the SQUID magnetic sensor. Thus, the protein was specific to the rabbit IgG, and the amount of the protein which was bound to the antibody was determined through the magnetic signal from the magnetic marker. FIG. 9 shows the results of the SQUID output measurements against the amount of the protein. The abscissa is the weight of the protein (pg), while the ordinate is the SQUID output (mΦ₀). As can be seen from the figure, there is a good relationship between the weight of the protein and the SQUID output. As it is possible for a SQUID sensor to make a measurement to a level of 0.1 mΦ₀ or lower, the figure indicates that the magnetic marker enables the measurement of the protein down to even an amount as low as about 0.2 pg.

### INDUSTRIAL UTILITY

As can be seen from the above explanation, the magnetic marker of the present invention enables the measurement of an immunoreaction (antigen-antibody) reaction with extremely high-sensitivity, and therefore is expected to make a large contribution in a number of fields, including medical fields current achieving rapid progress, by making it possible to measure biological substances which have been conventionally impossible to measure.

What is claimed is:

1. A magnetic marker composed of a magnetic fine particle and a polymer encapsulating the particle, for use in measuring an immunoreaction with a SQUID magnetic sensor, wherein the particle diameter of said magnetic fine particle is 20 to 40 nm and the external diameter of said magnetic marker is 40 to 100 nm, said polymer having carboxyl groups on the surface thereof.

2. The magnetic marker as claimed in claim 1, wherein the magnetic fine particle is composed of ferrite Fe₃O₄.

3. The magnetic marker as claimed in claim 2, wherein the polymer has, on the surface thereof, 500 to 5000 carboxyl groups per particle of the magnetic marker.

4. The magnetic marker as claimed in claim 3, wherein the polymer has, on the surface thereof, 2000 to 3000 carboxyl groups per particle of the magnetic marker.

5. A method for preparing a magnetic marker composed of a magnetic fine particle and a polymer encapsulating the particle, for use in measuring an immunoreaction with a SQUID magnetic sensor, wherein the particle diameter of the magnetic fine particle is 20 to 40 nm and the external diameter of the magnetic marker is 40 to 100 nm, said polymer having carboxyl groups on the surface thereof, which method comprises the steps of (i) causing the surface of a magnetic fine particle to adsorb a hydrophilic macromonomer having a polymerizable vinyl group at the terminal thereof and having a molecular weight of 500 to 1000, and then (ii) adding a monomer of a hydrophilic vinyl compound having carboxyl groups and a crosslinking agent for carrying out copolymerization reaction.

6. The method for preparing the magnetic marker as claimed in claim 5, wherein the macromonomer is polyvinylpyrrolidone, polyoxyethylene or polyacrylamide.
